(19) United States
(12) Patent Application Publication
(10) Pub. No.: US 2004/0235948 A1
(43) Pub. Date: Nov. 25, 2004

(54) Treatment of diabetic patients with omega-3-fatty acids

(75) Inventors: Rainer Oelze, Hannover (DE); Cees-Nico Verboom, Wietze (DE)

Correspondence Address: CROWELL & MORING LLP, INTELLECTUAL PROPERTY GROUP, P.O. BOX 14300, WASHINGTON, DC, 20044-4300, US

(73) Assignee: Solvay Pharmaceuticals GmbH, Hannover (DE)

(21) Appl. No.: 10/792,918

(22) Filed: Mar. 5, 2004

Related U.S. Application Data
(60) Provisional application No. 60/454,318, filed on Mar. 14, 2003.

(30) Foreign Application Priority Data
Mar. 5, 2003 (EP) 03 00 4792.2

(51) Int. Cl. A61K 31/22

(52) U.S. Cl. 514/549

(57) ABSTRACT

The use of essential fatty acids with a high content of eicosapentaenoic acid ethyl ester (EPA) and/or docosahexaenoic acid ethyl ester (DHA) for preventing cardiovascular events in patients with diabetes mellitus.

### CROSS REFERENCE TO RELATED APPLICATIONS

This application claims priority from prior U.S. provisional patent application No. 60/454,318, filed Mar. 14, 2003, the entire disclosure of which is incorporated herein by reference. Priority is also claimed based on European patent application no. EP 03 00 4792.2, filed Mar. 5, 2003.

### BACKGROUND OF THE INVENTION

This invention concerns the use of a pharmaceutical composition containing essential fatty acid ethyl esters originating from fish oils, in particular as a high concentration mixture of ethyl esters of (20:5ω 3) eicosapentaenoic acid (EPA) and (22:6ω 3) docosahexaenoic acid (DHA) in patients who suffer from diabetes.

It is well known that certain essential fatty acids contained in fish oil have a therapeutic effect in the prevention and treatment of cardiovascular disorders, such as in the treatment of hypertension, thrombosis, hypercholesterolemia, arteriosclerosis, cerebral infarction, prevention of sudden death in post myocardial infarction patients, improvement of endothelial function and hyperlipedemias.

U.S. Pat. No. 5,502,077, U.S. Pat. No. 5,656,667 and U.S. Pat. No. 5,698,594 can be cited as examples. The prevention of cardiovascular events, especially of mortality in patients who have survived the hospitalization phase of acute myocardial infarction (AMI) is described in published international patent application WO 00/48592.

The prior art cited above in particular provide knowledge about the utility of fatty acids belonging to the ω-3 family, more specifically (20:5ω 3) eicosapentaenoic acid (EPA) and (22:6ω 3) docosahexaenoic acid (DHA), in treating the above-mentioned disorders.

The fatty acid EPA, being a precursor of PGI3 and TxA3, exerts a platelet aggregation preventing effect and an anti-thombotic effect that can be ascribed to inhibition of cyclooxygenase (similar to the effect of aspirin) and/or to competition with arachidonic acid for this enzyme, with consequent reduction in the sythesis of PGE2 and TxA2, which are well known platelet aggregating agents.

On the other hand the fatty acid DHA is the most important component of cerebral lipids in man and furthermore, being a structural component of the platelet cell it intervenes indirectly in increasing platelet fluidity, thus playing an important role in antithombotic activity.

The international patent application WO 89/11521, the entire disclosure of which is incorporated herein by reference, describes in particular an industrial process for extracting mixtures with a high content in poly-unsaturated acids, including EPA and DHA and their ethyl esters, from animal and/or vegetable oils. Mixtures of fatty acids, especially EPA/DHA, obtained according to WO 89/11521, are reported to be particularly useful in the treatment of cardiovascular diseases.

However, current methods of treatment used in human therapy have been shown to be insufficient in patients who have a diabetes mellitus, in particular in those patients in whom it is desired to also prevent cardiovascular events. It is well known that patients with diabetes, in particular with diabetes mellitus, are at a substantially increased risk of cardiovascular events and death.

Therefore, there has remained a substantial need for improved and effective treatments with drugs, in particular for preventing these recurrences.

### SUMMARY OF THE INVENTION

It is therefore an object of the present invention to provide an improved method of treating diabetic patients.

Another object of the invention is to provide a method of treating patients suffering from diabetes mellitus to inhibit undesired cardiovascular events.

A further object of the invention is to provide a new method for preparing a pharmaceutical composition adapted for treating patients suffering from diabetes.

These and other objects are achieved in accordance with the present invention by providing a method of treating a patient with diabetes comprising administering to the patient a therapeutically effective amount of an essential fatty acid composition comprising greater than 25% by weight of at least one substance selected from the group consisting of eicosapentanoic acid ethyl ester (EPA) and docosahexaenoic acid ethyl ester (DHA).

In accordance with a further aspect of the invention, the objects are also achieved by providing a method of preparing a medicament suitable for treating a patient with diabetes comprising forming an essential fatty acid composition comprising greater than 25% by weight of at least one substance selected from the group consisting of eicosapentanoic acid ethyl ester (EPA) and docosahexaenoic acid ethyl ester (DHA) into a therapeutically administerable pharmaceutical dosage form.

This invention, therefore, suggests the novel use of essential fatty acids with a high content in EPA-ethyl ester or DHA-ethyl ester or a high concentration mixture thereof, in the preparation of a medicament useful for the treatment of patients suffering from diabetes. In particular, the invention is directed to preventing cardiovascular events in patients who have diabetes mellitus.

For ease of description “EPA-ethyl ester” and “DHA-ethyl ester” will be also be referred to here as “EPA” and “DHA”.

In particular this invention pertains to the use of essential fatty acids containing a mixture of eicosapentanoic acid ethyl ester (EPA) and docosahexaenoic acid ethyl ester (DHA) in the preparation of a medicament useful for the treatment of patients suffering from diabetes, preferably for preventing cardiovascular events in patients who have diabetes, where the content in EPA and DHA in such mixture is greater than 25% by weight.

An essential fatty acid with high content in EPA or DHA, according to the present invention, preferably contains more than 25% by weight, in particular from about 60 to about 100% of such ester. These compounds can be obtained by known methods.

In an essential fatty acid with a high concentration mixture of EPA and DHA, preferably such mixture has a content in EPA and DHA greater than 25% by weight, in particular from about 30 to about 100% by weight, preferably about 85% by weight. In the EPA/DHA mixture, EPA preferably is present in a percentage from about 40 to 60% by weight and DHA, preferably in a percentage from about 25 to about 45-50%.

In any case, the preferred EPA/DHA ratio in such EPA/DHA mixture is about 0.9/1.5.

Pharmacology

Diabetes mellitus has become an increasingly prevalent disease worldwide. The prevalence of diabetes is increasing rapidly and the number of individuals with type II diabetes (80-90% of all diabetic people) is depicted to reach 300 million in the year 2025, accounting for 5.4% of the global population. Furthermore, cardiovascular events are important contributors to morbidity and mortality in patients with diabetic disease. The risk of death from cardiovascular disease is in patients with diabetes two to six times that among persons without diabetes. Currently, over 50% of diabetic patients die from coronary heart disease. In contrast to non-diabetic people, coronary heart mortality has not declined in diabetic people. Type II diabetes eliminates the protective advantage of female sex against coronary heart disease mortality. The prognosis after a coronary heart disease event is poorer in diabetic people than in non-diabetic people. Within 1 year after an acute myocardial infarction, 44.2% of type II diabetic men and 36.9% of type II diabetic women die.

All manifestations of coronary heart disease are at least twice as common in patients with diabetes as in non-diabetic individuals. Moreover, close interrelationships between diabetes and cardiovascular disease, not least with coronary artery disease, have recently been elucidated. It has been demonstrated in a number of studies that 28% of patients with known coronary artery disease have diabetes, and as many as 70% of patients with acute coronary syndromes have abnormal glucose metabolism, either in the form of diabetes or impaired glucose tolerance. Major risk factors for coronary heart disease in patients with diabetes include:

1. unfavorable lipoprotein profile, characterized by increased serum triglycerides;

2. elevated blood pressure;

3. predisposition to formation of thrombosis, including the following manifestations: high concentrations of plasminogen activator-1 and cytokines;

4. impairment of endothelin-dependent vasodilatation;

5. cardiac autonomic impairment leading to decreased ischaemic pain perception, higher heart rate and decresed heart rate variability, which in turn increases the risk for sudden death.

The efficacy of the treatment according to the present invention is established by ample pre-clinical and clinical evidence:

1. EPA plus DHA induces a reduction in the levels of triglycerides and of very-low density lipoprotein cholesterol (VLDL) in patients with hypertriglyceremia;

2. EPA plus DHA does lower blood pressure in patients with hypertension;

3. Dietary EPA and DHA down-regulate gene expression of platelet-derived growth factor-A and of platelet-derived growth factor-B in human mononuclear cells;

4. Supplementation with EPA plus DHA mitigates the course of coronary atherosclerosis in patients with coronary heart disease;

5. EPA and DHA improves endothelial function in heart transplant recipients.

6. Experimental studies have shown that EPA and DHA are antiarrhythmic in several animal models, probably due to specific modulation of ion currents;

7. EPA and DHA increases heart rate variability in healthy volunteers and in survivors of a myocardial infarction;

8. EPA plus DHA decreases the incidence of sudden death in survivors of a myocardial infarction.

The foregoing evidence of reducing risk factors shows that the present invention provides a new and valuable therapeutic tool for treating diabetic patients, and in particular for preventing cardiovascular events in diabetic patients.

Accordingly, this invention also provides a method for treating diabetic patients, preferably patients with diabetes mellitus and in particular for preventing cardiovascular events in diabetic patients, preferably in patients with diabetes mellitus, comprising administering to such patient a therapeutically effective amount of a medicament containing essential fatty acids with a high content in EPA-ethyl ester or DHA-ethyl ester or a high concentration mixture thereof.

The essential fatty acids, according to the invention, can either have a high content, for instance more than 25% by weight, of EPA or DHA or a mixture thereof. However, EPA and DHA-ethyl ester are preferably present as a mixture thereof with a content of EPA and DHA greater than 25% by weight, in particular from about 30 to about 100% by weight, preferably about 85% by weight.

Based on the available evidence, according to a preferred aspect of the invention, the dosage of an essential fatty acid containing an EPA and DHA mixture with 85% by weight titer for oral administration to a patient may vary from about 0.7 gram to about 6 grams daily, preferably about 1 gram daily.

This amount of product as EPA and DHA mixture (or amount of EPA alone or DHA alone) may be administered in several divided doses throughout the day or preferably in a single administration, in order to achieve the desired blood level. Of course, it is at the discretion of the treating physician to adjust the quantity of product to be administered depending on the age, weight and general condition of the patient.

The medicament, e.g. in the form of a pharmaceutical composition, according to this invention can be prepared according to known methods in the art. The preferred route of administration is the oral one, however leaving alternative routes of administration, such as the parenteral route, to the discretion of the treating physician.

The following examples illustrate preferred formulations for oral administration, but are not intended to limit the scope of the invention in any way.

Gelatin Capsules

Capsules having the following composition and containing 1 g of active ingredient (EPA and DHA, 85% titer) per capsule are prepared according to known pharmaceutical techniques.

| Formulation 1: | | |
|---|---|---|
| EPA-ethyl ester | 525 | mg/capsule |
| DHA-ethyl ester | 315 | mg/capsule |
| d-alpha tocopherol | 4IU/capsule | |
| gelatin | 246 | mg/capsule |
| glycerol | 118 | mg/capsule |
| red iron oxide | 2.27 | mg/capsule |
| yellow iron oxide | 1.27 | mg/capsule |
| Formulation 2: | | |
| Ethyl esters of polyunsaturated fatty acids | 1000 | mg |
| with content in ethyl esters of w-3 poly- | | |
| unsaturated esters (eicosapentaenoic EPA, | | |
| docosahexaenoic DHA) | 850 | mg |
| d-1-α-tocopherol | 0.3 | mg |
| gelatin succinate | 233 | mg |
| glycerol | 67 | mg |
| sodium p-oxybenzoate | 1.09 | mg |
| sodium propyl p-oxobenzoate | 0.54 | mg |

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

What is claimed is:

1. A method of treating a patient with diabetes, said method comprising administering to said patient a therapeutically effective amount of an essential fatty acid composition comprising greater than 25% by weight of at least one substance selected from the group consisting of eicosapentanoic acid ethyl ester (EPA) and docosahexaenoic acid ethyl ester (DHA).

2. A method according to claim 1, wherein said fatty acid composition comprises greater than 25% by weight of a mixture of EPA and DHA.

3. A method according to claim 1, wherein said fatty acid composition comprises greater than 25% by weight of EPA.

4. A method according to claim 1, wherein said fatty acid composition comprises greater than 25% by weight of DHA.

5. A method according to claim 1, wherein said essential fatty acid composition is administered to said patient in an amount effective to inhibit cardiovascular events in a patient with diabetes.

6. A method according to claim 1, wherein said patient is a patient suffering from diabetes mellitus.

7. A method according to claim 2, wherein said fatty acid composition has a content of EPA and DHA lying in the range from about 30 to 100% by weight.

8. A method according to claim 7, wherein said fatty acid composition has a content of EPA and DHA of about 85% by weight.

9. A method according to claim 3, wherein said fatty acid composition has a content of EPA lying in the range from about 60% by weight to 100% by weight.

10. A method according to claim 4, wherein said fatty acid composition has a content of DHA lying in the range from about 60% by weight to 100% by weight.

11. A method according to claim 1, wherein said fatty acid composition is administered orally.

12. A method according to claim 11, wherein said fatty acid composition is administered at a daily dosage of from about 0.7 g to about 6 g.

13. A method according to claim 12, wherein said fatty acid composition is administered once daily.

14. A method according to claim 12, wherein said fatty acid composition is administered in multiple subdoses at intervals throughout the day.

15. A method according to claim 2, wherein the ratio of EPA/DHA in said mixture is about 0.9/1.5.

16. A method of preparing a medicament suitable for treating a patient with diabetes, said method comprising forming an essential fatty acid composition comprising greater than 25% by weight of at least one substance selected from the group consisting of eicosapentanoic acid ethyl ester (EPA) and docosahexaenoic acid ethyl ester (DHA) into a therapeutically administerable pharmaceutical dosage form.

17. A method according to claim 16, wherein said fatty acid composition comprises greater than 25% by weight of a mixture of EPA and DHA.

18. A method according to claim 17, wherein said fatty acid composition has a content of EPA and DHA lying in the range from about 30 to 100% by weight.

19. A method according to claim 18, wherein said fatty acid composition has a content of EPA and DHA of about 85% by weight.

20. A method according to claim 16, wherein said fatty acid composition comprises greater than 25% by weight of EPA or DHA.

21. A method according to claim 20, wherein aid fatty acid composition has a content of EPA or DHA lying in the range from about 60% to 100% by weight.

22. A method according to claim 17, wherein the ratio of EPA to DHA in said mixture is about 0.9 to 1.5.

23. A method according to claim 16, wherein said dosage form is an orally administerable dosage form.

24. A method according to claim 23, wherein said dosage form is obtained by filling from about 0.7 g to about 6 g of said fatty acid composition into a gelatin capsule.

25. A method according to claim 24, wherein said fatty acid composition is admixed with at least one pharmaceutical auxiliary substance.
